(19) United States
(12) Patent Application Publication
(10) Pub. No.: US 2025/0092216 A1
(43) Pub. Date: Mar. 20, 2025

(54) DECOMPOSITION OF POLYOLEFINS

(71) Applicant: OXFORD UNIVERSITY INNOVATION LIMITED, Oxford (GB)

(72) Inventors: Shik Chi Edman TSANG, Oxford (Oxfordshire) (GB); Haokun WANG, Oxford (Oxfordshire) (GB)

(21) Appl. No.: 18/727,146

(22) PCT Filed: Jan. 19, 2023
(86) PCT No.: PCT/GB2023/050089

§ 371 Date: Jul. 8, 2024

(30) Foreign Application Priority Data
Jan. 19, 2022 (GB) 2200664.7

(51) Int. Cl. C08J 11/16

(52) U.S. Cl. CPC C08J 11/16

(57) ABSTRACT

The present invention relates to a process for the catalytic decomposition of a polyolefin. More particularly, the present invention relates hydrocarbon-aided catalytic decomposition of a polyolefin using an aluminosilicate.

### INTRODUCTION

The present invention relates to a process for the catalytic decomposition of a polyolefin. More particularly, the present invention relates to hydrocarbon-aided catalytic decomposition of a polyolefin using an aluminosilicate.

### BACKGROUND OF THE INVENTION

Since the fabrication of the first fully synthetic polymer in 1907, plastics have gradually become an indispensable part of modern life due to their cheap, durable, convenient and versatile properties (Vollmer, et al., 2021). These desirable properties have seen plastics become a key material in many fields such as packaging, electronics and construction. With the demand for plastics high, an appropriate waste management strategy is required but has not yet been established, largely due to the low recyclability of plastics (Chen, et al., 2021; Zhang, et al., 2021). Currently, it is estimated that more than 400 million tonnes of plastics are generated annually, and this number is projected to reach 1200 million by 2050 (Liu, et al., 2021). It is also estimated that 79% of the plastic produced annually end up in landfill sites with only ˜9% recycled for further use (Dutta & Gupta, 2021). This situation has been amplified by the global coronavirus pandemic due to the increased consumption of single-use personal protective equipment (PPE), such as surgical masks and gloves (Haque, et al., 2021).

Mechanical recycling is a traditional plastic waste disposing method but is highly energy-intensive and results in low-value products which exhibit poor durability (Kunwar, et al., 2017). Incineration is also a common technique in which energy from plastics can be recovered by combustion for further utilisation. This process, however, can produce carcinogenic gasses due to incomplete combustion, posing threats to health and the environment (Jiao, et al., 2021).

Chemical recycling, on the other hand, is seen as an attractive alternative for converting waste plastics into more valuable products. The conversion of waste plastic back to monomers can provide a useful feedstock for forming valuable products such as gasoline and diesel (Czajczynska, et al., 2018). Pyrolysis is a typical chemical recycling process and involves thermal cracking of long chain polyolefins into smaller hydrocarbon molecules at high temperature (up to 800° C.) (Aisien, et al., 2021). Various attempts at lowering the temperature and improving the product fuel quality using a catalyst have been reported (Dutta & Gupta, 2021) as well as modifications to other parameters such as reactor design and plastic-to-catalyst ratio (Idumah, 2021). However, up until now no viable route has been established with many attempts suffering scale-up problems such as cost, energy-consumption and wide product distributions. Thus, there remains a need for an efficient and selective process for the decomposition of waste plastics into valuable products.

The present invention was devised with the foregoing in mind.

### SUMMARY OF THE INVENTION

According to an aspect of the present invention there is provided a process for the catalytic decomposition of a polyolefin, the process comprising a step of contacting a polyolefin with:
(a) an aluminosilicate, and
(b) a hydrocarbon
   at a temperature of at least 200° C. and under an inert atmosphere,
   wherein the hydrocarbon comprises 2-20 carbon atoms and the aluminosilicate comprises a plurality of Brønsted acid sites.

The process for the catalytic decomposition of a polyolefin may be a process for the preparation of one or more hydrocarbons (e.g. a mixture of hydrocarbons) found in gasoline.

The one or more hydrocarbons prepared by the process may each be a C5-C12 hydrocarbon.

### DETAILED DESCRIPTION OF THE INVENTION

The term “(m-nC)” or “(m-nC) group” used alone or as a prefix, refers to any group having m to n carbon atoms.

The term “alkyl” as used herein refers to straight or branched chain alkyl moieties, typically having 1, 2, 3, 4 or 5 carbon atoms. This term includes reference to groups such as methyl, ethyl, propyl (n-propyl or isopropyl), butyl (n-butyl, sec-butyl or tert-butyl), pentyl and the like. Most suitably, an alkyl may have 1, 2 or 3 carbon atoms.

The term “alkenyl” as used herein refers to straight or branched chain alkenyl moieties, typically having 2, 3, 4 or 5 carbon atoms. The term includes reference to alkenyl moieties typically containing 1 or 2 carbon-carbon double bonds (C═C). This term includes reference to groups such as ethenyl (vinyl), propenyl (allyl), butenyl and pentenyl, as well as both the cis and trans isomers thereof.

The term “alkynyl” as used herein refers to straight or branched chain alkynyl moieties, typically having 2, 3, 4 or 5 carbon atoms. The term includes reference to alkynyl moieties typically containing 1 or 2 carbon-carbon triple bonds (C═C). This term includes reference to groups such as ethynyl, propynyl, butynyl and pentynyl.

The term “aliphatic” as used herein refers to straight, branched and non-aromatic ring moieties. Aliphatic compounds may be saturated (i.e., without a C═C double bond) or unsaturated (i.e., comprising a C═C double bond). Aliphatic compounds are typically alkanes (n-, iso-, neo- and cyclo-) such as hexane, heptane, octane, nonane, decane and may (or may not) also include one or more (e.g., 1, 2, 3 or 4) heteroatoms from selected nitrogen, oxygen, sulfur and chlorine. Aliphatic compounds may also be alkenes and alkynes (n-, iso-, neo- and cyclo-) and may (or may not) also include one or more (e.g., 1, 2, 3 or 4) heteroatoms from selected nitrogen, oxygen, sulfur and chlorine.

The term “aromaticity” as used herein refers to the presence of an aromatic ring system typically comprising 6, 7, 8, 9 or 10 ring carbon atoms. An aromatic ring system is often phenyl, but may be a polycyclic ring system having two fused rings, at least one of which is aromatic. This term includes reference to groups such as phenyl, naphthyl and the like.

The term “heteroaromaticity” as used herein refers to the presence of an aromatic ring system incorporating one or more (e.g., 1, 2 or 3) ring heteroatoms selected from nitrogen, oxygen and sulfur. A heteroaromatic ring system is often monocyclic, but may be a polycyclic ring system having two fused rings, at least one of which is heteroaromatic. Typically, the heteroaromatic ring system is a 5- or 6-membered ring. Typically, the heteroaromatic ring system will contain up to 3 ring heteroatoms (e.g., nitrogen), more usually up to 2, for example a single ring heteroatom.

The term “substituted” as used herein in reference to a moiety means that one or more (e.g., 1, 2, 3 or 4) of the hydrogen atoms in said moiety are replaced independently of each other by the corresponding number of the described substituents. The term “optionally substituted” as used herein means substituted or unsubstituted.

It will, of course, be understood that substituents are only at positions where they are chemically possible, the person skilled in the art being able to decide (either experimentally or theoretically) without inappropriate effort whether a particular substitution is possible.

Throughout the entirety of the description and claims of this specification, where subject matter is described herein using the term “comprise” (or “comprises” or “comprising”), the same subject matter instead described using the term “consist of” (or “consists of” or “consisting of”) or “consist essentially of” (or “consists essentially of” or “consisting essentially of”) is also contemplated.

Throughout the entirety of the description and claims of this specification, the singular encompasses the plural unless the context otherwise requires. In particular, where the indefinite article is used, the specification is to be understood as contemplating plurality as well as singularity, unless the context requires otherwise.

Features described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any of the specific embodiments recited herein. The invention extends to any novel one, or any novel combination, of the features disclosed in this specification (including any accompanying claims, abstract and drawings), or to any novel one, or any novel combination, of the steps of any method or process so disclosed.

As described hereinbefore, an aspect of the invention provides a process for the catalytic decomposition of a polyolefin, the process comprising a step of contacting a polyolefin with:
(a) an aluminosilicate, and
(b) a hydrocarbon
   at a temperature of at least 200° C. and under an inert atmosphere,
   wherein the hydrocarbon comprises 2-20 carbon atoms and the aluminosilicate comprises a plurality of Brønsted acid sites (BAS).

Through rigorous investigations, the inventors have devised a vastly improved process for the selective and efficient decomposition of waste plastics. In particular, the inventors have found that an aluminosilicate catalyst with a plurality of BAS, when used in combination with particular hydrocarbons, allows polyolefins to be readily decomposed under mild conditions, with the reaction offering a particular selectivity for valuable hydrocarbon fractions, such as gasoline (a mixture of C5-12 compounds). The inventors have shown that this process is an industrially viable technique, which, due to the mild conditions involved (e.g., low temperature and short reaction duration), is workable on a large scale. Furthermore, the inventors have found that not only can the process of the present invention improve the selectivity of polyolefin decomposition to more valuable products, the process also offers advantages in terms of recyclability and costs.

It will be understood that the term “polyolefin” used herein refers to a polymer comprising repeating units formed from the polymerisation of olefin monomers. The polyolefin may therefore comprise repeating units formed from the polymerisation of ethylene monomers, propylene monomers, ethylene terephthalate monomers, vinyl chloride monomers, styrene monomers, or a combination of two or more thereof. Thus, the polyolefin may comprise polyethylene (PE), polypropylene (PP), polyethylene terephthalate (PET), polyvinyl chloride (PVC), polystyrene (PS), or a combination of two or more thereof. As discussed hereinbefore, the process of the present invention is particularly useful in the conversion of waste plastics. Accordingly, the polyolefin may be provided in the form of a plastic (e.g., a waste plastic). Thus, the present invention also provides a process for the catalytic decomposition of a polyolefin provided in the form of a plastic, wherein the process comprises the step of contacting a polyolefin with an aluminosilicate and a hydrocarbon in accordance with the aspect of the present invention. Suitably the plastic is waste plastic.

The polyolefin may comprise greater than 60 wt % of PE, PP or a combination thereof. Suitably, the polyolefin comprises greater than 70 wt % of PE, PP or a combination thereof. More suitably, the polyolefin comprises greater than 80 wt % of PE, PP or a combination thereof. Yet more suitably, the polyolefin comprises greater than 90 wt % of PE, PP or a combination thereof. Yet even more suitably, the polyolefin comprises greater than 95 wt % of PE, PP or a combination thereof. Yet still even more suitably, the polyolefin comprises greater than 99 wt % of PE, PP or a combination thereof. In embodiments wherein the polyolefin comprises greater than 60 wt %, 70 wt %, 80 wt %, 90 wt %, 95 wt % or 99 wt % PE, PP or a combination thereof, the remainder of the polyolefin may comprise other repeating units. For example, the remainder of the polyolefin may comprise repeating units formed from the polymerisation of vinyl chloride, styrene, ethylene terephthalate, phenol, formaldehyde, ethylene glycol, acetonitrile or a combination of two or more thereof.

In some embodiments, the polyolefin is PE, PP or a combination thereof (e.g., the polyolefin consists of PE, PP or a combination thereof). It will be understood that in embodiments wherein the polyolefin comprises a combination of PE and PP, the combination may be in the form of a physical mixture (i.e., a sample comprising both PE and PP) or a copolymer (i.e., block, random or alternate copolymer) of PE and PP.

It will be further understood that the term “polyolefin” used herein encompasses modified polyolefins such as cross-linked polyolefins (e.g., cross-linked polyethylene (PEX)) and ethylene propylene diene monomer (EPDM) rubber) and branched polyolefins.

In embodiments, the polyolefin is selected from the group consisting of high density polyethylene (HDPE), low density polyethylene (LDPE), linear low-density polyethylene (LLDPE), high density polypropylene (HDPP), low density polypropylene (LDPP), linear low-density polypropylene (LLDPP) and a combination of two or more thereof. Suitably, the polyolefin is selected from the group consisting of HDPE, LDPE, HDPP and a combination of two or more thereof. In an embodiment, the polyolefin is selected from the group consisting of HDPE, LDPE, HDPP and LDPE/HDPP/HDPP (i.e., a combination of LDPE, HDPP and HDPP). In an embodiment, the polyolefin is HDPE.

The inventors have found that the presence of aluminosilicates possessing a plurality of BAS significantly improves the effects of the hydrocarbon in promoting the decomposition of a polyolefin. The term “aluminosilicate” will be familiar to one of skill in the art. Aluminosilicates are solid, inorganic compounds comprising Al, Si and 0 atoms arranged as SiO₄ and AlO₄ tetrahedra. The Al, Si and 0 atoms in the aluminosilicate may vary in their degree of structural order such that the aluminosilicate may be crystalline (e.g., a zeolite) or amorphous (e.g., SiO₂—Al₂O₃). Without wishing to be bound by theory, the inventors have hypothesised that, during decomposition of a polyolefin, the aluminosilicate and the hydrocarbon work synergistically to boost the rate of C—C bond cleavage in a polyolefin to rapidly form valuable products such as C5-12 gasoline fractions. It is believed that this effect is partly due to the decomposition of the polyolefin taking place over BAS present in/on the aluminosilicate. The aluminosilicate may therefore be considered a catalyst in the process of the invention. The aluminosilicate may be used in an activated (e.g., calcined) form. The activated form of the aluminosilicate may be prepared by subjecting the aluminosilicate to a step of ion exchange with a salt (e.g., an ammonium salt), before a step of calcining the aluminosilicate. Calcination may involve thermally treating the aluminosilicate, e.g. at a temperature of 300-600° C.

The BAS of the aluminosilicate may be present on the aluminosilicate (i.e., on the surface of an aluminosilicate) or may be present in the aluminosilicate (i.e., in the pores of an aluminosilicate). Thus, it may be that the aluminosilicate is porous (i.e., comprises a plurality of pores) or is non-porous. In embodiments wherein the aluminosilicate comprises a plurality of pores, it may be that the BAS are located within the plurality of pores. The decomposition of the polyolefin, which takes place over BAS, may therefore take place within the plurality of pores of the aluminosilicate.

As discussed hereinbefore, the aluminosilicate may be a zeolite. The zeolite may be of the FAU framework type. The phrase “FAU framework” used in the context of the aluminosilicate is known in the art and will be understood to mean an aluminosilicate compound that belongs to the structure code FAU defined by the International Zeolite Association (IZA). The typical FAU zeolite framework comprises a plurality of pores which are defined by the number of ring atoms forming the perimeter of each pore. Suitably, the plurality of pores of the zeolite comprise 12-membered ring channels. Furthermore, in embodiments wherein the aluminosilicate is a zeolite, it may be that the zeolite is doped (i.e., one more dopant atoms replace an Al, Si and/or 0 atom in the zeolite framework). Suitably, the zeolite is doped with one or more atoms selected from the group consisting of B and N. More suitably, the zeolite is of the FAU framework type and is doped with one or more atoms selected from the group consisting of B and N.

The aluminosilicate (e.g., a zeolite) may have a plurality of pores. The plurality of pores may each have a diameter of 0.65-0.95 nm. Due to the diameter of each of the pores, the aluminosilicate may be considered to be microporous (i.e., comprising pores with pore diameters less than 2 nm). Suitably, the plurality of pores each have a diameter of 0.70-0.90 nm. More suitably, the plurality of pores each have a diameter of 0.72-0.85 nm. Yet more suitably, the plurality of pores each have a diameter of 0.75-0.80 nm.

A specific type of aluminosilicate with FAU framework is a Y-type zeolite which is acidified (i.e., the aluminosilicate comprises a plurality of BAS). Suitably, the aluminosilicate is selected from the group consisting of Y zeolite (e.g., Y(3.5), Y(5), Y(12), Y(30), Y(60) and Y(80)) and ultrastable Y (USY) zeolite, each of which having a plurality of BAS. The presence of a plurality of BAS in/on the aluminosilicate may be indicated by the prefix “H” (i.e., HY(3.5), HY(5), HY(12) etc.) to denote that the aluminosilicate comprises a plurality of BAS. It will be understood that additional charge-balancing ions, such as metal cations (e.g., Na⁺), may also form part of the aluminosilicate framework. Accordingly, the aluminosilicate may be selected from the group consisting of HY zeolite (e.g., HY(3.5), HY(5), HY(12), HY(30), HY(60) and HY(80)) and ultrastable HY (USY) zeolite (where H denotes that the aluminosilicate comprises a plurality of BAS). In an embodiment, the aluminosilicate is selected from the group consisting of HY(3.5), HY(5), HY(12), HY(30), HY(60), HY(80) and ultrastable HY (USY) zeolite.

As will be clear from the above discussion, the acidity of the aluminosilicate is of importance in the process of the present invention. In particular, the presence of a plurality of BAS in/on the aluminosilicate, which can be controlled by the SiO₂/Al₂O₃ ratio (i.e., the molar ratio of Si to Al atoms), promotes the selective decomposition of polyolefins. Suitably, the aluminosilicate has a SiO₂/Al₂O₃ ratio of 2-90. More suitably, the aluminosilicate has a SiO₂/Al₂O₃ ratio of 3-70. Yet more suitably, the aluminosilicate has a SiO₂/Al₂O₃ ratio of 5-50. Yet even more suitably, the aluminosilicate has a SiO₂/Al₂O₃ ratio of 8-30.

The aluminosilicate may have a specific surface area (calculated in accordance with the Brunauer-Emmett-Teller (BET) theory) of 200-1000 m²/g. Suitably, the aluminosilicate has a specific surface area of 400-800 m²/g. More suitably, the aluminosilicate has a specific surface area of 500-700 m²/g. In particular embodiments, the aluminosilicate has a specific surface area of 600-650 m²/g.

In an embodiment, the aluminosilicate has a SiO₂/Al₂O₃ ratio of 2-90 and a specific surface area of 400-800 m²/g. In a particular embodiment, the aluminosilicate has a SiO₂/Al₂O₃ ratio of 5-50 and a specific surface area of 500-700 m²/g.

It may be that the aluminosilicate is amorphous. The aluminosilicate may be amorphous Si₂O/Al₂O₃ having a plurality of BAS. Suitably, the aluminosilicate is amorphous Si₂O/Al₂O₃(H) (where, as discussed above, H denotes that the aluminosilicate comprises a plurality of BAS). The amorphous aluminosilicate may have one or more properties selected from: an amount of Al of 10-16 wt %, a specific surface area of 500-700 m²g⁻¹, a pore volume of 0.65-0.85 cm³g⁻¹ and a SiO₂/Al₂O₃ ratio of 6-10. For example, the amorphous aluminosilicate may possess all of the aforementioned properties. A particular example of an amorphous aluminosilicate is acidified SiO₂—Al₂O₃ Grade 135. It will be understood that the features discussed herein in relation to the aluminosilicate will apply to both crystalline (e.g., HY and ultrastable HY) and amorphous (e.g., Si₂O/Al₂O₃(H)) aluminosilicates where chemically possible. Alternatively, the aluminosilicate may be mesoporous Si₂O/Al₂O₃ having a plurality of BAS.

In an embodiment, the aluminosilicate is selected from the group consisting of Y zeolite (e.g., Y(3.5), Y(5), Y(12), Y(30), Y(60) and Y(80)), ultrastable Y zeolite, and amorphous SiO₂/Al₂O₃, each of which having a plurality of BAS. In a particular embodiment, the aluminosilicate is selected from the group consisting of HY zeolite (e.g., HY(3.5), HY(5), HY(12), HY(30), HY(60) and HY(80)), ultrastable HY zeolite, and amorphous SiO₂/Al₂O₃(H). Suitably, the aluminosilicate is selected from the group consisting of HY(30), ultrastable HY zeolite and amorphous SiO₂/Al₂O₃(H).

The aluminosilicate may comprise one or more transition metal promoters. The one or more transition metal promoter may be selected from the group consisting of W, Re, Pt, Sn, Ir, Co and a combination of two or more thereof. The transition metal promoter may be present as an oxide (e.g., a tungstate), or a carbide (e.g., Ir/C) of any of these metals. Suitably, the transition metal promoter is selected from the group consisting of W, Re, Pt/Sn (i.e., a mixture of Pt and Sn), Ir and Pt/Co (i.e., a mixture of Pt and Co). The use of one or more transition metal promoters may be particularly useful when the hydrocarbon is an aliphatic compound.

Suitably, the aluminosilicate comprises 0.001-20 wt % of the one or more transition metal promoter. More suitably, the aluminosilicate comprises 0.005-15 wt % of the one or more transition metal promoter. Even more suitably, the aluminosilicate comprises 0.01-10 wt % of the one or more transition metal promoter.

In an embodiment, the polyolefin comprises greater than 60 wt % of PE, PP or a combination thereof and the aluminosilicate has a SiO₂/Al₂O₃ ratio of 2-90. In a particular embodiment, the polyolefin comprises greater than 80 wt % of PE, PP or a combination thereof and the aluminosilicate has a SiO₂/Al₂O₃ ratio of 5-50.

In an embodiment, the polyolefin is selected from the group consisting of HDPE, LDPE, LLDPE, HDPP, LDPP, LLDPP and a combination of two or more thereof and the aluminosilicate is selected from the group consisting of Y zeolite (e.g., Y(3.5), Y(12), Y(30), Y(60) and Y(80)), ultrastable Y zeolite, and amorphous SiO₂/Al₂O₃, each of which having a plurality of BAS.

The presence of a hydrocarbon in the process of the present invention leads to an efficient, selective and reusable process for converting waste plastics into more valuable products, such as gasoline hydrocarbon fractions. The inventors have hypothesised that the hydrocarbon promotes the decomposition of polyolefins by inducing β-scission of C—C bonds and cross metathesis under mild conditions. The hydrocarbon may have a molecular weight of less than 250 g mol⁻¹. Suitably, the hydrocarbon has a molecular weight of less than 225 g mol⁻¹. More suitably, the hydrocarbon has a molecular weight of less than 200 g mol⁻¹. Yet more suitably, the hydrocarbon has a molecular weight of less than 175 g mol⁻¹. Yet even more suitably, the hydrocarbon has a molecular weight of less than 150 g mol⁻¹.

In some embodiments the polyolefin comprises greater than 60 wt % of PE, PP or a combination and the hydrocarbon has a molecular weight of less than 200 g mol⁻¹.

The hydrocarbon comprises 2-20 carbon atoms in total. The hydrocarbon may comprise 5-20 carbon atoms. The hydrocarbon may comprise 5-18 carbon atoms. Suitably, the hydrocarbon comprises 5-16 carbon atoms. More suitably, the hydrocarbon comprises 5-14 carbon atoms. Yet more suitably, the hydrocarbon comprises 6-12 carbon atoms. Yet even more suitably, the hydrocarbon comprises 6-10 carbon atoms.

In various embodiments, the hydrocarbon comprising 2-20 carbon atoms is an aromatic compound comprising 5-20 carbon atoms. An aromatic compound will be understood as being a hydrocarbon compound having aromaticity or heteroaromaticity as defined hereinbefore. Aromatic compounds described herein comprise an aromatic or heteroaromatic ring system that is substituted (e.g., with 1, 2, 3 or 4 substituents) or unsubstituted. Suitably, the aromatic compound comprising 5-20 carbon atoms is a monocyclic aromatic ring (e.g., benzene) or a bicyclic aromatic ring system (e.g., naphthalene), any ring of which is optionally substituted. Possible substituents that may be present are (1-5C)alkyl, (2-5C)alkenyl and/or (2-5C)alkynyl. Suitably, each substituent is independently (1-3C)alkyl (e.g., methyl and/or isopropyl). The inventors have hypothesised that when the hydrocarbon possesses (hetero)aromaticity, the hydrocarbon alkylates the polyolefin followed by β-scission of a C—C bond to form an alkyl-substituted aromatic compound. It is believed that alkylation of the polyolefin boosts the rate of C—C bond cleavage in the polyolefin at mild conditions, suggesting that the hydrocarbon can act as a molecular tweezer by scavenging alkyl moieties from the polyolefin.

In some embodiments, the aluminosilicate has a SiO₂/Al₂O₃ ratio of 5-50 and the hydrocarbon is an aromatic compound comprising 5-20 carbon atoms. Suitably the aromatic compound comprising 5-20 carbon atoms is a monocyclic aromatic ring (e.g., benzene) or a bicyclic aromatic ring system (e.g., naphthalene), any ring of which is optionally substituted with one, two, three or four substituents independently selected from (1-3C)alkyl substituents.

In some embodiments, the hydrocarbon comprising 2-20 carbon atoms is benzene that is optionally substituted with one or more substituents independently selected from (1-5C)alkyl, (2-5C)alkenyl and (2-5C)alkynyl. Suitably, the hydrocarbon is benzene that is optionally substituted with one, two, three or four substituents independently selected from (1-3C)alkyl. More suitably, the hydrocarbon is benzene that is optionally substituted with one, two, three or four substituents independently selected from methyl, ethyl and isopropyl. In embodiments, the hydrocarbon is benzene that is optionally substituted with one, two, three or four substituents independently selected from methyl and isopropyl. Most suitably, the hydrocarbon is selected from the group consisting of benzene, toluene, xylene, cumene, mesitylene, 1,2,4,5-tetramethyl benzene and naphthalene. In a particular embodiment, the hydrocarbon is selected from the group consisting of benzene, toluene, xylene, cumene, mesitylene and 1,2,4,5-tetramethyl benzene.

In some embodiments, the hydrocarbon has a molecular weight of less than 200 g mol⁻¹ and is an aromatic compound comprising 5-20 carbon atoms. In a particular embodiment, the hydrocarbon has a molecular weight of less than 150 g mol⁻¹ and is benzene that is optionally substituted with one, two, three or four substituents independently selected from methyl and isopropyl.

In various other embodiments, the hydrocarbon comprising 2-20 carbon atoms is an aliphatic compound. Suitably, the hydrocarbon is an aliphatic compound comprising 2-18 carbon atoms. More suitably, the hydrocarbon is an aliphatic compound comprising 2-16 carbon atoms. Yet more suitably, the hydrocarbon is an aliphatic compound comprising 2-14 carbon atoms. Yet even more suitably, the hydrocarbon is an aliphatic compound comprising 2-12 carbon atoms. In embodiments, the hydrocarbon is an aliphatic compound comprising 2-10 carbon atoms. In embodiments, the hydrocarbon is an aliphatic compound comprising 4-10 carbon atoms. In embodiments, the hydrocarbon is an aliphatic compound comprising 6-10 carbon atoms. In a particular embodiment, the hydrocarbon is an aliphatic compound comprising 8-10 carbon atoms (e.g. decane). The inventors have hypothesised that when the hydrocarbon is aliphatic, the hydrocarbon works in tandem with the aluminosilicate to first dehydrogenate the aliphatic hydrocarbon (when the aliphatic hydrocarbon does not comprise any unsaturated bonds) and the polyolefin (i.e., to form one or more 7r-bonds in the hydrocarbon and the polyolefin) before the dehydrogenated hydrocarbon undergoes cross-metathesis with the polyolefin, thereby forming at least two new hydrocarbon fractions. The new hydrocarbon fractions are fragments of the original polyolefin and are thought to undergo a final hydrogenation step to form C5-C12 compounds such as gasoline. Suitably, the hydrocarbon is a saturated aliphatic compound (i.e., the hydrocarbon is an alkane comprising 2 to 20 carbon atoms). It will be understood than in embodiments wherein the hydrocarbon is an alkane comprising 2 to 20 carbon atoms, the hydrocarbon may be the n-, iso-, neo- or cyclo-structural form thereof. Alternatively, the hydrocarbon is an unsaturated aliphatic compound (i.e., the hydrocarbon is an alkene or an alkyne comprising 2 to 20 carbon atoms), such an ethene or propene.

In embodiments, the hydrocarbon comprising 2-20 carbon atoms is an aliphatic compound and the aluminosilicate comprises one or more transition metal promoters (e.g. W, Re, Pt, Sn, Ir and/or Co, including their oxides and carbides). Suitably, the hydrocarbon is a saturated aliphatic compound comprising 2-12 carbon atoms (e.g. decane).

The hydrocarbon, when aliphatic, may be selected from the group consisting of ethene, propene, butene, ethane, propane, butane, pentane, hexane, heptane, octane, nonane and decane. Suitably, the hydrocarbon is ethene, propene or decane. Most suitably, the hydrocarbon, when aliphatic, is decane.

Thus, the hydrocarbon may be selected from the group consisting of ethene, propene, butene, ethane, propane, butane, pentane, hexane, heptane, octane, nonane, decane, benzene, toluene, xylene, cumene, mesitylene, 1,2,4,5-tetramethyl benzene and naphthalene. In a particular embodiment, the hydrocarbon is selected from the group consisting of decane, benzene, toluene, xylene, cumene, mesitylene and 1,2,4,5-tetramethyl benzene.

In some embodiments, the polyolefin comprises greater than 80 wt % of PE, PP or a combination thereof; the aluminosilicate is selected from the group consisting of Y zeolite (e.g., Y(3.5), Y(12), Y(30), Y(60) and Y(80)), ultrastable Y zeolite, and amorphous SiO₂/Al₂O₃, each of which having a plurality of BAS; and the hydrocarbon is selected from the group consisting of hexane, heptane, octane, nonane, decane, benzene, toluene, xylene, cumene, mesitylene, 1,2,4,5-tetramethyl benzene and naphthalene.

In some embodiments, the polyolefin is selected from the group consisting of HDPE, LDPE, LLDPE, HDPP, LDPP, LLDPP and a combination of two or more thereof; the aluminosilicate has a SiO₂/Al₂O₃ ratio of 5-50; and the hydrocarbon is benzene that is optionally substituted with one, two, three or four (1-3C)alkyl substituents.

In some embodiments, the hydrocarbon is a saturated aliphatic compound comprising 2-12 carbon atoms (e.g. decane) and the aluminosilicate is amorphous SiO₂/Al₂O₃, having a plurality of BAS, comprising W (or its oxide or carbide) and Pt/Sn or Ir/C as transitional metal promoters. In such embodiments, the aluminosilicate may be provided as a combination of a first amorphous SiO₂/Al₂O₃ aluminosilicate, having a plurality of BAS, comprising W (or its oxide or carbide) as transition metal promoter, and a second amorphous SiO₂/Al₂O₃ aluminosilicate, having a plurality of BAS, comprising Pt/Sn or Ir/C as transition metal promoter.

The process of the present invention can be conducted at relatively mild conditions when compared to known processes, which typically consume a vast amount of energy due to the high temperatures traditionally required for polyolefin decomposition. Owing to the synergistic benefits of the aluminosilicate and the hydrocarbon, it is possible for the reaction to proceed at temperatures below those typical for polyolefin decomposition.

The step of contacting a polyolefin with an aluminosilicate and a hydrocarbon may be conducted at a temperature of 200-600° C. Suitably, the step of contacting a polyolefin with an aluminosilicate and a hydrocarbon is conducted at a temperature of 300-600° C. More suitably, the step of contacting a polyolefin with an aluminosilicate and a hydrocarbon is conducted at a temperature of 300-500° C. Yet more suitably, the step of contacting a polyolefin with an aluminosilicate and a hydrocarbon is conducted at a temperature of 300-450° C. Yet even more suitably, the step of contacting a polyolefin with an aluminosilicate and a hydrocarbon is conducted at a temperature of 300-400° C. In some embodiments, the step of contacting a polyolefin with an aluminosilicate and a hydrocarbon is conducted at a temperature of 300-390° C. In a particular embodiment, the step of contacting a polyolefin with an aluminosilicate and a hydrocarbon is conducted at a temperature of 300-350° C.

Suitably, the weight ratio of aluminosilicate to hydrocarbon is 1: (0.1-10). More suitably, the weight ratio of aluminosilicate to hydrocarbon is 1: (0.5-5).

The step of contacting a polyolefin with an aluminosilicate and a hydrocarbon is conducted under an inert atmosphere. Suitably, the inert atmosphere comprises at least one inert gas selected from the group consisting of nitrogen, hydrogen and argon. The inert atmosphere may comprise 10-40 bar nitrogen. Suitably, the inert atmosphere comprises 20-30 bar nitrogen. The inert atmosphere may comprise 10-40 bar hydrogen. Suitably, the inert atmosphere comprises 20-30 bar hydrogen.

In some embodiments, the step of contacting a polyolefin with an aluminosilicate and a hydrocarbon is conducted at a temperature of 300-600° C. and under an inert atmosphere comprising at least one inert gas selected from the group consisting of nitrogen, hydrogen and argon. In a particular embodiment, the step of contacting a polyolefin with an aluminosilicate and a hydrocarbon is conducted at a temperature of 300-400° C. and under an inert atmosphere comprising 20-30 bar nitrogen or 20-30 bar hydrogen.

The following numbered statements 1 to 80 are not claims, but instead describe particular aspects and embodiments of the invention:

1. A process for the catalytic decomposition of a polyolefin, the process comprising a step of contacting a polyolefin with:
(a) an aluminosilicate, and
(b) a hydrocarbon
   at a temperature of at least 200° C. and under an inert atmosphere,
   wherein the hydrocarbon comprises 2-20 carbon atoms and the aluminosilicate comprises a plurality of Brønsted acid sites.
   2. The process of statement 1, wherein the polyolefin comprises repeating units formed from the polymerisation of ethylene monomers, propylene monomers, ethylene terephthalate monomers, vinyl chloride monomers, styrene monomers, or a combination of two or more thereof.
   3. The process of any one of statements 1 or 2, wherein the polyolefin comprises PE, PP, PET, PVC, PS, or a combination of two or more thereof.
   4. The process of any one of statements 1, 2 or 3, wherein the polyolefin is provided in the form of a plastic.
   5. The process of any one of the preceding statements, wherein the polyolefin is provided in the form of a waste plastic.
   6. The process of any one of the preceding statements, wherein the polyolefin comprises greater than 60 wt % of PE, PP or a combination thereof.
   7. The process of any one of the preceding statements, wherein the polyolefin comprises greater than 70 wt % of PE, PP or a combination thereof.
   8. The process of any one of the preceding statements, wherein the polyolefin comprises greater than 80 wt % of PE, PP or a combination thereof.
   9. The process of any one of the preceding statements, wherein the polyolefin comprises greater than 90 wt % of PE, PP or a combination thereof.
   10. The process of any one of the preceding statements, wherein the polyolefin comprises greater than 95 wt % of PE, PP or a combination thereof.
   11. The process of any one of the preceding statements, wherein the polyolefin comprises greater than 99 wt % of PE, PP or a combination thereof.
   12. The process of any one of the preceding statements, wherein the polyolefin is PE, PP or a combination thereof.
   13. The process of any one of the preceding statements, wherein the polyolefin is selected from the group consisting of HDPE, LDPE, LLDPE, HDPP, LDPP, LLDPP and a combination of two or more thereof.
   14. The process of any one of the preceding statements, wherein the polyolefin is selected from the group consisting of HDPE, LDPE, HDPP and a combination of two or more thereof.
   15. The process of any one of the preceding statements, wherein the polyolefin is selected from the group consisting of HDPE, LDPE, HDPP and LDPE/HDPP/HDPP.
   16. The process of any one of the preceding statements, wherein the aluminosilicate is crystalline or amorphous.
   17. The process of any one of the preceding statements, wherein the aluminosilicate is a zeolite.
   18. The process of any one of the preceding statements, wherein the aluminosilicate is a zeolite of the FAU framework type.
   19. The process of statement 17 or 18, wherein the zeolite comprises a plurality of pores.
   20. The process of statement 19, wherein the plurality of pores comprise 12-membered ring channels.
   21. The process of any one of statements 19 or 20, wherein the plurality of pores each have a diameter of 0.65-0.95 nm.
   22. The process of any one of statements 19, 20 or 21, wherein the plurality of pores each have a diameter of 0.70-0.90 nm.
   23. The process of any one of statements 19 to 22, wherein the plurality of pores each have a diameter of 0.72-0.85 nm.
   24. The process of any one of statements 19 to 23, wherein the plurality of pores each have a diameter of 0.75-0.80 nm.
   25. The process of any one of the preceding statements, wherein the aluminosilicate is a Y-type zeolite.
   26. The process of any one of the preceding statements, wherein the aluminosilicate has a SiO₂/Al₂O₃ ratio of 2-90.
   27. The process of any one of the preceding statements, wherein the aluminosilicate has a SiO₂/Al₂O₃ ratio of 3-70.
   28. The process of any one of the preceding statements, wherein the aluminosilicate has a SiO₂/Al₂O₃ ratio of 5-50.
   29. The process of any one of the preceding statements, wherein the aluminosilicate has a SiO₂/Al₂O₃ ratio of 8-30.
   30. The process of any one of the preceding statements, wherein the aluminosilicate has a specific surface area of 200-1000 m²/g.
   31. The process of any one of the preceding statements, wherein the aluminosilicate has a specific surface area of 400-800 m²/g.
   32. The process of any one of the preceding statements, wherein the aluminosilicate has a specific surface area of 500-700 m²/g.
   33. The process of any one of the preceding statements, wherein the aluminosilicate has a specific surface area of 600-650 m²/g.
   34. The process of any one of the preceding statements, wherein the aluminosilicate is selected from the group consisting of Y zeolite (e.g., Y(3.5), Y(5), Y(12), Y(30), Y(60) and Y(80)), ultrastable Y zeolite, and amorphous SiO₂/Al₂O₃, each of which having a plurality of BAS.
   35. The process of any one of the preceding statements, wherein the aluminosilicate is selected from the group consisting of Y(30), ultrastable Y zeolite and amorphous SiO₂/Al₂O₃, each of which having a plurality of BAS.
   36. The process of any one of the preceding statements, wherein the aluminosilicate comprises one or more transition metal promoters.
   37. The process of statement 36, wherein the one or more transition metal promoter is selected from the group consisting of W, Re, Pt, Sn, Ir, Co and a combination of two or more thereof.
   38. The process of any one of statements 36 or 37, wherein the transition metal promoter is selected from the group consisting of W, Re and Pt/Sn.
   39. The process of any one of statements 36, 37 or 38, wherein the aluminosilicate comprises 0.001-20 wt % of the one or more transition metal promoter.
   40. The process of any one of statements 36 to 39, wherein the aluminosilicate comprises 0.005-15 wt % of the one or more transition metal promoter.
   41. The process of any one of statements 36 to 40, wherein the aluminosilicate comprises 0.01-10 wt % of the one or more transition metal promoter.
   42. The process of any one of the preceding statements, wherein the hydrocarbon has a molecular weight of less than 250 g mol⁻¹.
   43. The process of any one of the preceding statements, wherein the hydrocarbon has a molecular weight of less than 225 g mol⁻¹.
   44. The process of any one of the preceding statements, wherein the hydrocarbon has a molecular weight of less than 200 g mol⁻¹.
   45. The process of any one of the preceding statements, wherein the hydrocarbon has a molecular weight of less than 175 g mol⁻¹.
   46. The process of any one of the preceding statements, wherein the hydrocarbon has a molecular weight of less than 150 g mol⁻¹.
   47. The process of any one of the preceding statements, wherein the hydrocarbon comprises 5-18 carbon atoms.
   48. The process of any one of the preceding statements, wherein the hydrocarbon comprises 5-16 carbon atoms.
   49. The process of any one of the preceding statements, wherein the hydrocarbon comprises 5-14 carbon atoms.
   50. The process of any one of the preceding statements, wherein the hydrocarbon comprises 6-12 carbon atoms.
   51. The process of any one of the preceding statements, wherein the hydrocarbon comprises 6-10 carbon atoms.
   52. The process of any one of statements 1 to 46, wherein the hydrocarbon is an aromatic compound comprising 5-20 carbon atoms.
   53. The process of statement 52, wherein the aromatic compound comprising 5-20 carbon atoms is a monocyclic aromatic ring (e.g., benzene) or a bicyclic aromatic ring system (e.g., naphthalene), any ring of which is optionally substituted with one or more substituents independently selected from (1-5C)alkyl, (2-5C)alkenyl and (2-5C)alkynyl.
   54. The process of statement 53, wherein the one or more substituents are each independently selected from (1-4C)alkyl, (2-4C)alkenyl and (2-4C)alkynyl.
   55. The process of statement 53, wherein the one or more substituents are one, two, three or four substituents, each independently selected from(1-3C)alkyl.
   56. The process of statement 53, wherein the one or more substituents are one, two, three or four substituents, each independently selected from methyl, ethyl and isopropyl.
   57. The process of statements 53, wherein the one or more substituents are one, two, three or four methyl substituents.
   58. The process of any one of statements 1 to 52, wherein the hydrocarbon is benzene that is optionally substituted with one or more substituents independently selected from (1-5C) alkyl, (2-5C)alkenyl and (2-5C)alkynyl.
   59. The process of statement 58, wherein the hydrocarbon is benzene that is optionally substituted with one, two, three or four (1-3C)alkyl substituents.
   60. The process of statement 58, wherein the hydrocarbon is benzene that is optionally substituted with one, two, three or four substituents independently selected from methyl, ethyl and isopropyl.
   61. The process of statement 58, wherein the hydrocarbon is benzene that is optionally substituted with one, two, three or four substituents independently selected from methyl and isopropyl.
   62. The process of any one of statements 1 to 51, wherein the hydrocarbon is an aliphatic compound.
   63. The process of any one of statements 1 to 51, wherein the hydrocarbon is selected from the group consisting of ethene, propene, butene, ethane, propane, butane, pentane, hexane, heptane, nonane, decane, benzene, toluene, xylene, cumene, mesitylene, 1,2,4,5-tetramethyl benzene and naphthalene.
   64. The process of any one of statements 1 to 51, wherein the hydrocarbon is selected from the group consisting of decane, benzene, toluene, xylene, cumene, mesitylene and 1,2,4,5-tetramethyl benzene.
   65. The process of any one of the preceding statements, wherein the step of contacting a polyolefin with an aluminosilicate and a hydrocarbon is conducted at a temperature of 200-600° C.
   66. The process of any one of the preceding statements, wherein the step of contacting a polyolefin with an aluminosilicate and a hydrocarbon is conducted at a temperature of 300-600° C.
   67. The process of any one of the preceding statements, wherein the step of contacting a polyolefin with an aluminosilicate and a hydrocarbon is conducted at a temperature of 300-500° C.
   68. The process of any one of the preceding statements, wherein the step of contacting a polyolefin with an aluminosilicate and a hydrocarbon is conducted at a temperature of 300-450° C.
   69. The process of any one of the preceding statements, wherein the step of contacting a polyolefin with an aluminosilicate and a hydrocarbon is conducted at a temperature of 300-400° C.
   70. The process of any one of the preceding statements, wherein the step of contacting a polyolefin with an aluminosilicate and a hydrocarbon is conducted at a temperature of 300-390° C.
   71. The process of any one of the preceding statements, wherein the step of contacting a polyolefin with an aluminosilicate and a hydrocarbon is conducted at a temperature of 300-350° C.
   72. The process of any one of the preceding statements, wherein the weight ratio of aluminosilicate to hydrocarbon is 1: (0.1-10).
   73. The process of any one of the preceding statements, wherein the weight ratio of aluminosilicate to hydrocarbon is 1: (0.5-5).
   74. The process of any one of the preceding statements, wherein the inert atmosphere comprises an inert gas selected from the group consisting of nitrogen, hydrogen and argon.
   75. The process of any one of the preceding statements, wherein the inert atmosphere comprises 10-40 bar nitrogen.
   76. The process of any one of the preceding statements, wherein the inert atmosphere comprises 20-30 bar nitrogen.
   77. The process of any one of the preceding statements, wherein the inert atmosphere comprises 10-40 bar hydrogen.
   78. The process of any one of the preceding statements, wherein the inert atmosphere comprises 20-30 bar hydrogen.
   79. The process of any one of the preceding statements, wherein the aluminosilicate is a zeolite and is doped with one or more atoms selected from the group consisting of B and N.
   80. The process of any one of the preceding statements, wherein the aluminosilicate is a zeolite of the FAU framework type and is doped with one or more atoms selected from the group consisting of B and N.

### EXAMPLES

One or more examples of the invention will now be described, for the purpose of illustration only, with reference to the accompanying figures:

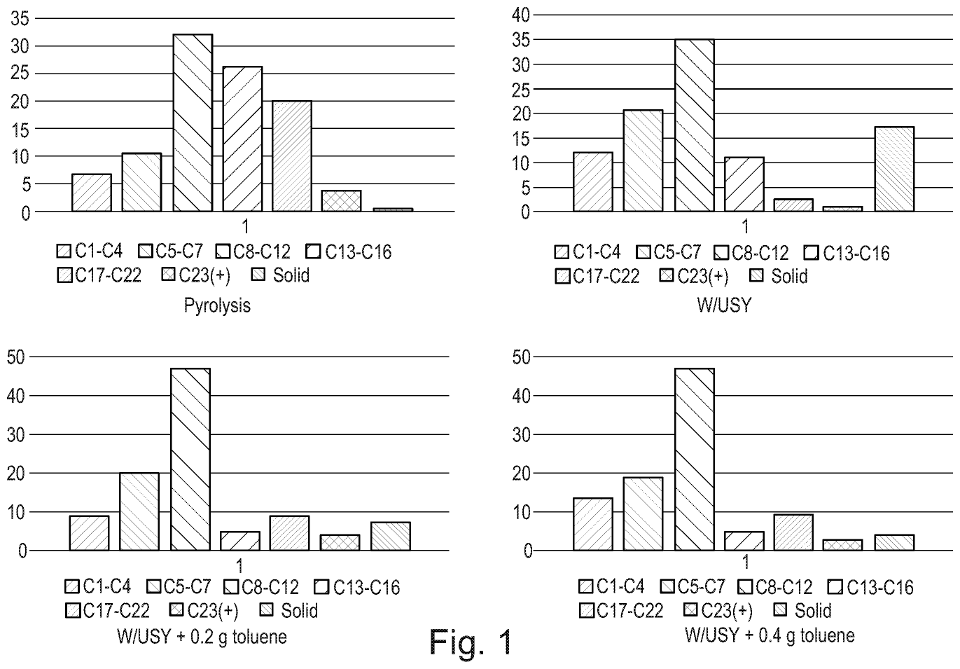

FIG. 1. The final product distribution from 2 g HDPE at 330° C., 20 bar N₂ after 4h over 0.2 g aluminosilicate catalyst (with and without varying amounts of toluene) compared to pyrolysis with no aluminosilicate catalyst.

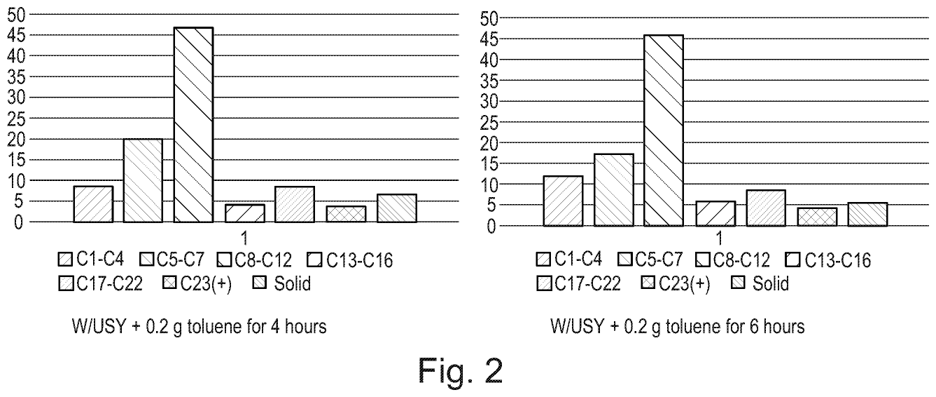

FIG. 2. The final product distribution from 2 g HDPE at 330° C., 20 bar N₂ after 4 h and after 6h over 0.2 g aluminosilicate catalyst.

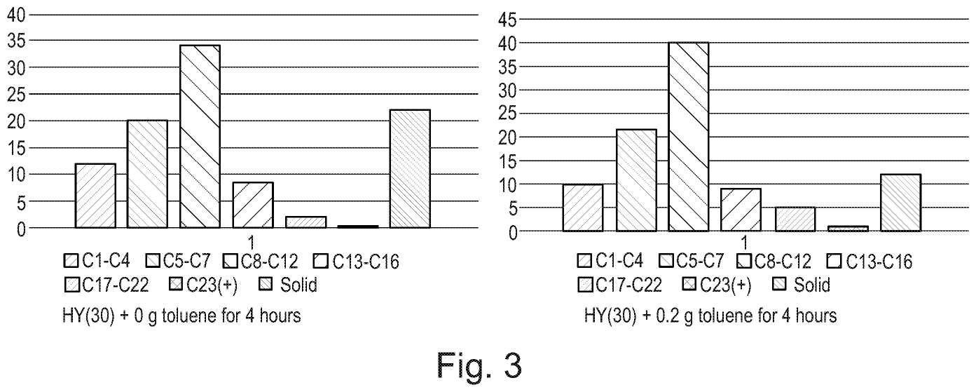

FIG. 3. The final product distribution from 2 g HDPE at 330° C., 30 bar N₂ after 4h over 0.2 g HY (30) catalyst.

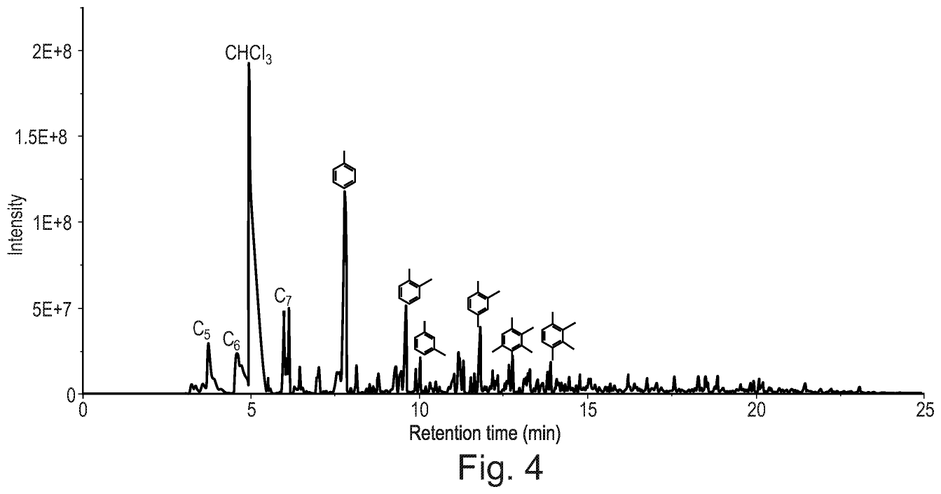

FIG. 4. Gas chromatography (GC) pattern of liquid products in CHCl₃. Reaction conditions: 0.2 g toluene, at 330° C., 20 bar N₂ after 4h over 0.2 g HY (30) catalyst.

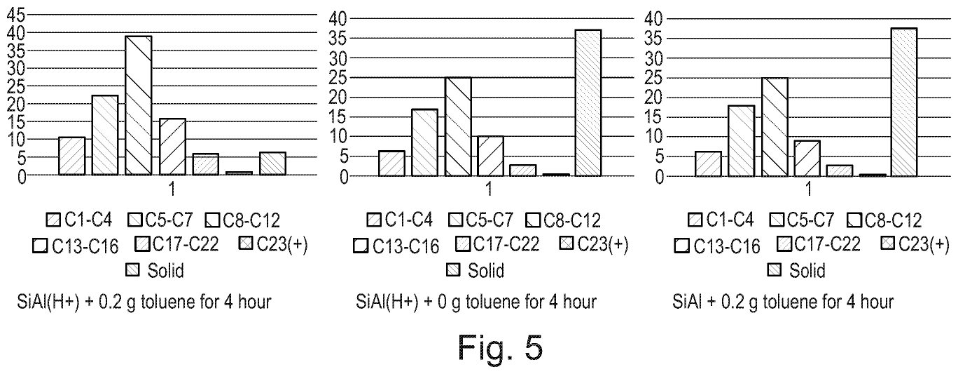

FIG. 5. The final product distribution from 2 g HDPE at 300° C., 20 bar N₂ after 4h over 0.2 g SiO₂—Al₂O₃ (with and without an acid wash) catalyst.

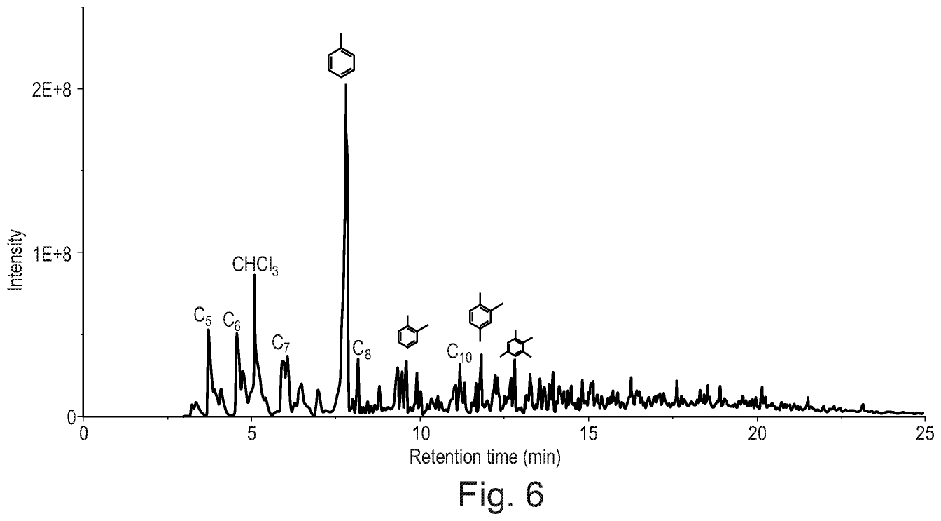

FIG. 6. GC pattern of liquid products in CHCl₃. Reaction conditions: 0.2 g toluene, 2 g HDPE at 300° C., 20 bar N₂ after 4h over 0.2 g SiO₂—Al₂O₃ catalyst.

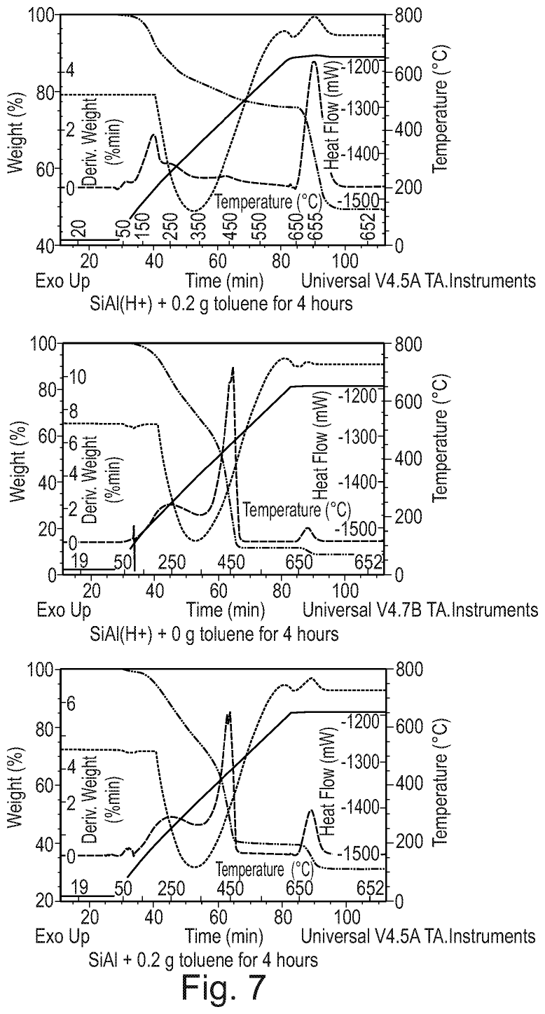

FIG. 7. Differential scanning calorimetry (DSC) and thermogravimetric analysis (TGA) for SiO₂—Al₂O₃ catalyst (solid residue). Ramp 10° C. min⁻¹ from room temperature to 650° C. in N₂ flow and then isothermal in air flow.

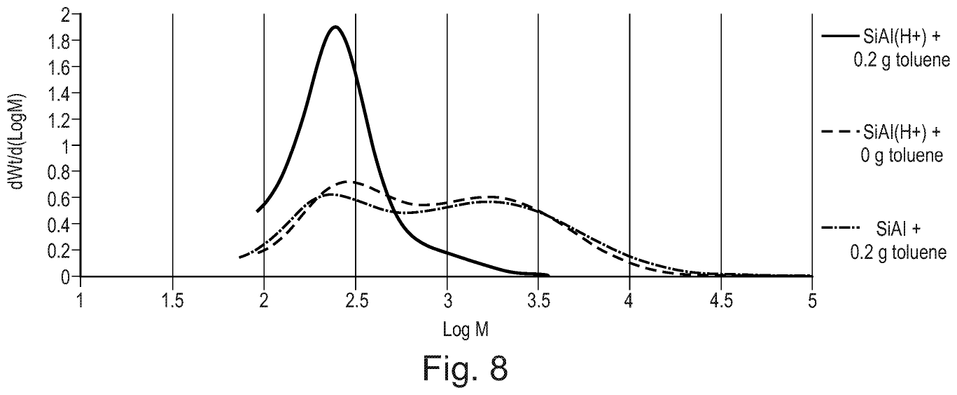

FIG. 8. Gel permeation chromatography (GPC) results for the SiO₂—Al₂O₃ catalyst (solid residue). 2 g HDPE at 300° C., 20 bar N₂ after 4h.

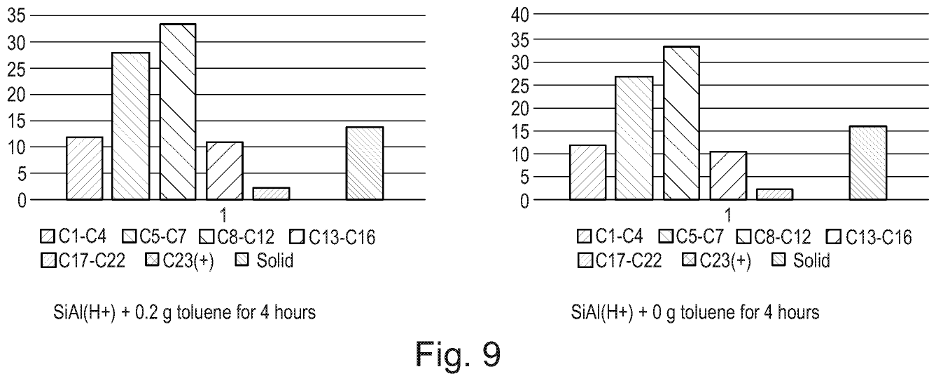

FIG. 9. The final product distribution from 2 g HDPE at 330° C., 20 bar N₂ after 4h over 0.2 g SiO₂—Al₂O₃ (with acid wash) with and without toluene.

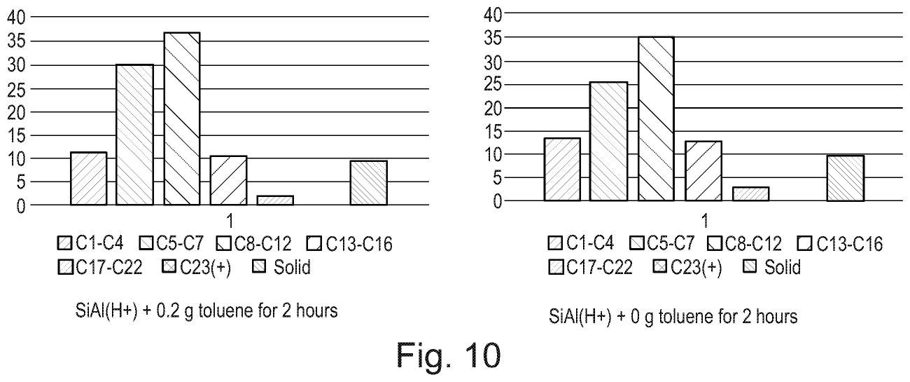

FIG. 10. The final product distribution from 2 g HDPE at 330° C., 20 bar N₂ after 2h over 0.2 g SiO₂—Al₂O₃ (with acid wash) with and without toluene.

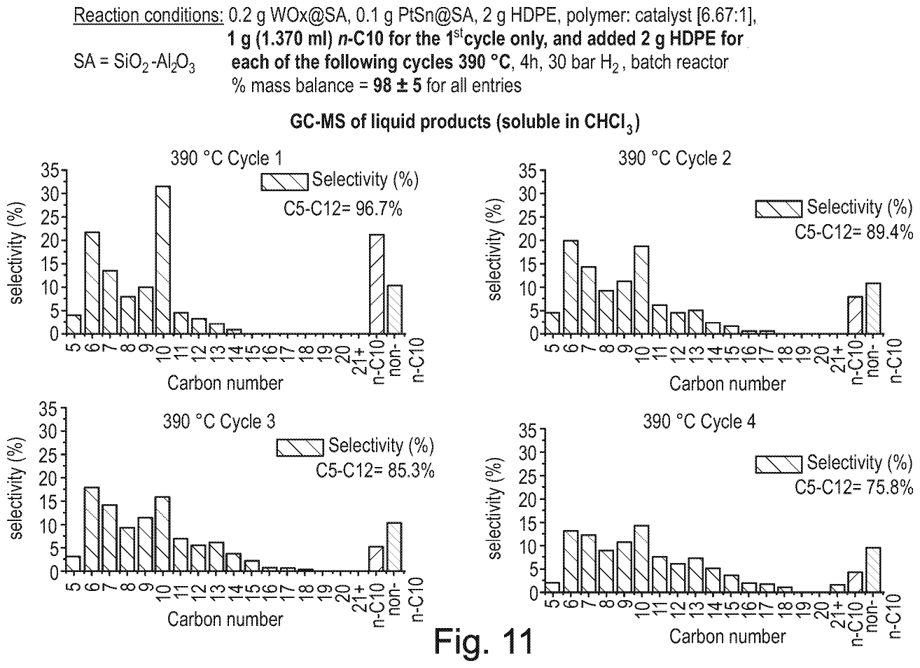

FIG. 11. The final product distribution from 0.2 g WO×@SiO₂—Al₂O₃, 0.1 g PtSn@SiO₂—Al₂O₃, 2 g HDPE, 1 g (1.37 mL) n-decane for the 1st cycle only (polymer: catalyst ratio of 6.67:1). 2 g HDPE was added for each subsequent cycle at 390° C., 4 h, 30 bar H₂, batch reactor. % mass balance=98±5 for all entries.

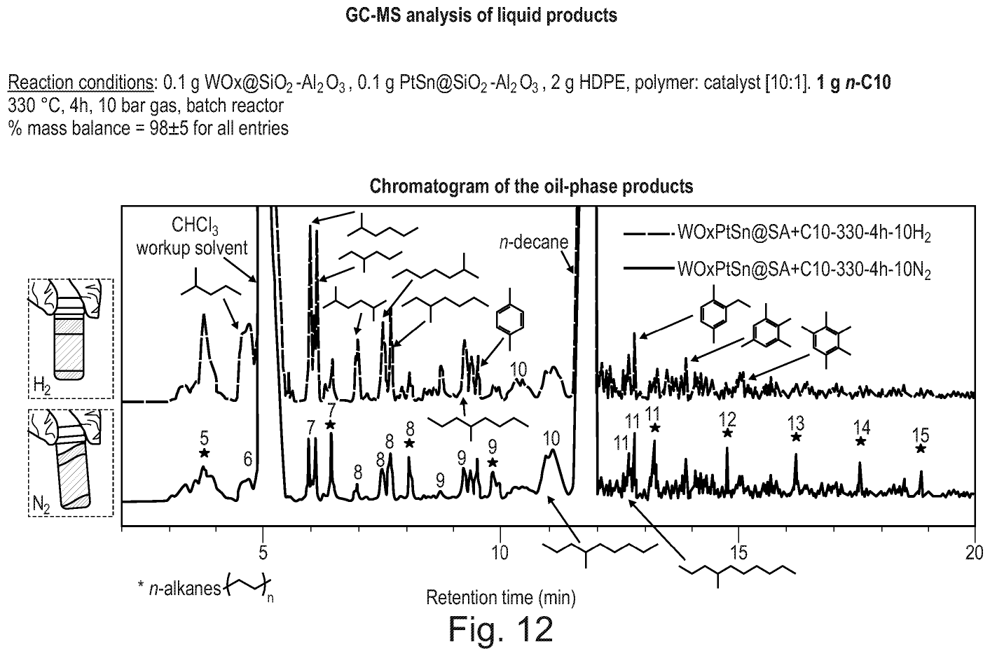

FIG. 12. The gas chromatography-mass spectrometry (GC-MS) analysis of the final product from 0.2 g WO×@SiO₂—Al₂O₃, 0.1 g PtSn@SiO₂—Al₂O₃, 2 g HDPE, 1 g (1.37 mL) n-decane for the 1st cycle only (polymer: catalyst ratio of 6.67:1). 2 g HDPE was added for each subsequent cycle at 390° C., 4 h, 30 bar H₂, batch reactor. % mass balance=98±5 for all entries.

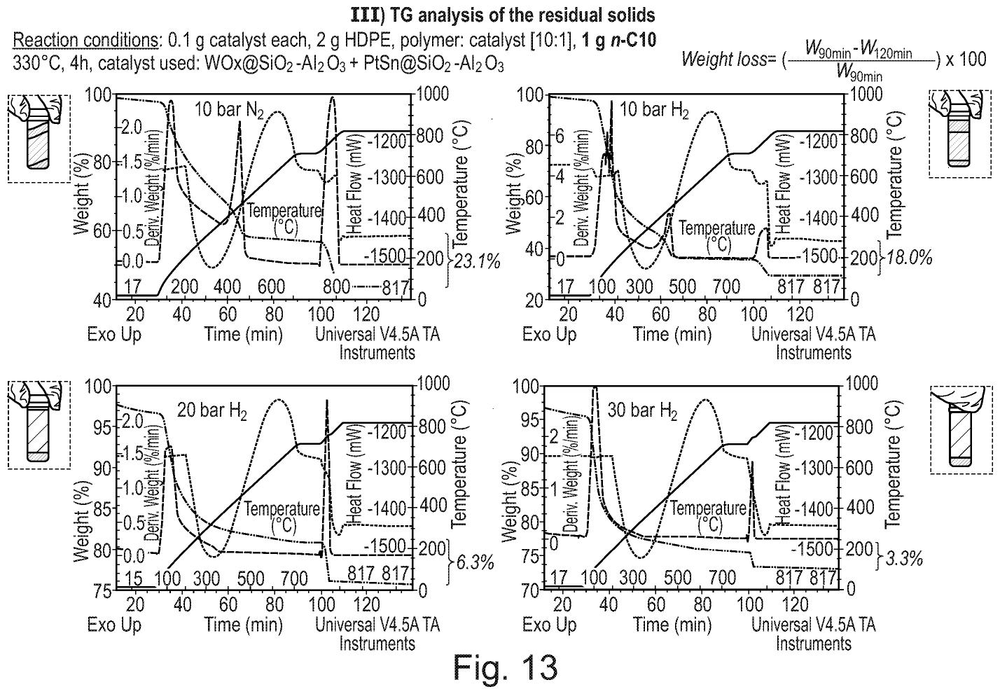

FIG. 13. DSC and TGA analysis for the WO×@SiO₂—Al₂O₃ and PtSn@SiO₂—Al₂O₃ catalyst mixture after polyolefin decomposition.

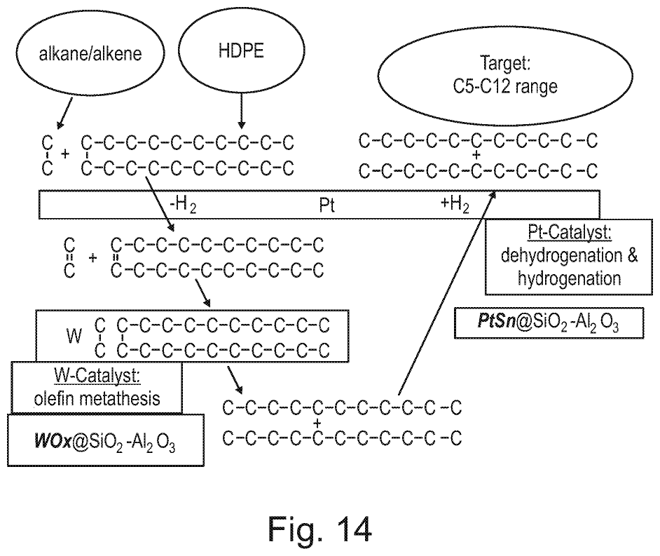

FIG. 14. n-decane promoted decomposition of HDPE via molecular averaging through dehydrogenation, exhaustive cross-metathesis and hydrogenation.

### MATERIALS AND METHODS

HDPE (Mw, 88707 g/mol, Mn, 10794 g/mol) was received from SCG Ltd. The polyolefin was used without further treatment. Aluminosilicate catalysts SiO₂—Al₂O₃ Grade 135, HY (30) zeolite and USY zeolite were purchased from Sigma-Aldrich. Chloroform (puriss. p.a., reag. ISO, reag. Ph. Eur., 99.0-99.4% GC), toluene (anhydrous, 99.8%) and ammonium nitrate (ACS reagent, >98%) were also purchased from Sigma-Aldrich.

### Catalyst Pre-Treatment and Cation Ion Exchange

All aluminosilicate catalysts except proton-exchanged SiO₂—Al₂O₃ were pre-treated at 400° C. (ramp 5° C. min⁻¹) in air flow (30 mL min⁻¹) for 3h prior to use. Proton exchanged SiO₂—Al₂O₃, denoted SiO₂—Al₂O₃ (H+), was synthesised as follows: ammonium nitrate was weighed and mixed with DI water to form 15 wt % ammonium nitrate solution. 3 g of SiO₂—Al₂O₃ was then put into the ammonium nitrate solution and heated to 80° C. for 4 h under stirring. After the hot suspension was cooled to room temperature, it was centrifuged and the solid was further washed with DI water three times. The washed solid was dried at 105° C. overnight before it was calcined in air flow at 600° C. for 4h prior to use.

### Catalytic Test

The catalytic test was carried out in a 50 mL autoclave, wherein 2 g of polyolefin was mixed with the aluminosilicate catalyst and toluene. Typically, 0.2 g of aluminosilicate catalyst and 0.2 g of toluene were introduced under 20 bar nitrogen after the air inside the autoclave was removed. The autoclave was heated to the target temperature in 1.5h. Meanwhile, the reactor was kept stirring with a glassy coated stirrer. After the heating program was finished, the temperature of the autoclave was allowed to cool to room temperature.

The gaseous product was analysed by GC while the liquid phase product was analysed by GC-MS (Agilent GC-MS 6890). The liquid-solid mixture was separated by centrifugation before the collected liquid product was injected into the GC-MS for analysis. Generally, chloroform was used to help with collection of the liquid-solid mixture from the autoclave. The solid from the centrifugation was dried in vacuo at 80° C. overnight before it was weighed at room temperature.

The final product was typically divided into three phases (gas, liquid, and solid). The mass of the autoclave including the stirrer was initially weighed using a balance with a measuring range and accuracy of 5 kg±0.05 g. The gas mass was verified by the pressure difference of the autoclave before and after the reaction and then calculated according to the Ideal Gas Law. The liquid-solid mixture mass was measured by the weight difference between the autoclave after discharging the gas and the empty autoclave (stirrer included). The solid mass was obtained by removing the weight of the aluminosilicate catalyst added and the weight of the liquid was verified by deducting the weight of the solid residue and aluminosilicate catalyst added from the liquid-solid mixture. The fraction of the desired compound (compound I) in the product and mass balance was performed as follows (initial toluene added was deducted from final calculation):

$$The fraction of compound I = \frac{The mass of compound I}{Total mass collected - mass of catalyst} \times 100 \% \qquad ( 1 )$$

$$Mass Balance = \frac{Total mass collected - mass of catalyst}{Total mass input - mass of catalyst} \times 100 \% \qquad ( 2 )$$

### Results and Discussion

TABLE 1
Product distribution in mass and mass balance in 2 g HDPE at
330° C., 20 bar N₂, 4 h. Gasoline (C5-C12); Diesel (C9-C22).

| Catalyst | Toluene | C1-C4 | Gasoline | Diesel | C23+ | Solid | Mass balance |
|---|---|---|---|---|---|---|---|
| — | — | 6.72% | 42.52% | 72.64% | 3.72% | 0.53% | 84.4% |
| W/USY | 0.2 g | 8.86% | 67.11% | 52.06% | 3.89% | 7.10% | 89.1% |
| Re/USY | 0.2 g | 10.01% | 57.43% | 53.10% | 3.49% | 12.88% | 93.5% |
| Re/USY | 0.4 g | 10.00% | 58.44% | 58.57% | 5.71% | 8.23% | 86.0% |
| W/USY | 0.4 g | 13.56% | 65.62% | 53.32% | 2.70% | 3.98% | 94.2% |
| W/USY (6 hours) | 0.2 g | 11.90% | 63.34% | 53.40% | 4.53% | 5.66% | 92.6% |
| W/USY | — | 12.37% | 56.02% | 41.13% | 0.85% | 17.36% | 96.8% |

The main objective was to optimize gasoline production (C5-C12) from waste plastics without excessive contamination with heavy hydrocarbon products over the aluminosilicate catalysts. As seen from the catalyst screening in Table 1 and the product distribution in FIG. 1, pyrolysis without the presence of an aluminosilicate catalyst can be observed to give nearly complete decomposition of HDPE (0.53% residue left) when heated to 330° C. The reaction yielded 42.52% gasoline products (sum of C5-C7 and C8-C12) and showed contamination with gaseous hydrocarbons (6.72%) (C1-C4) as well as heavy hydrocarbons (50.23%). Using the W/USY catalyst without promotion with a hydrocarbon (toluene) promoter increased the gaseous hydrocarbon (12.37%) and gasoline (56.02%) fractions at the expense of heavy hydrocarbons. Unconverted plastic residues (i.e., plastic which has not decomposed) remained relatively high (17.36%), however, indicating that some of the plastic was trapped in the porous structure of the aluminosilicate catalyst. Interestingly, adding 0.2 g of toluene into the reaction, enhanced the conversion of unconverted plastic (reduced to 7.1%) and increased gasoline production (67.11%). Without wishing to be bound by theory, the inventors believe that the presence of a hydrocarbon promoter improves the rate of C—C bond cleavage in the polyolefin, thereby producing more valuable hydrocarbon fractions such as gasoline. Increasing the toluene content from 0.2 g to 0.4 g increased the gaseous hydrocarbon fraction (13.56%) at the expense of heavy hydrocarbons to account for a total gas and gasoline content of ˜79%. Using Re/USY showed a similar trend but with a slightly lower gasoline fraction. Similar observations were also obtained when increasing the reaction time (Table 2 and FIG. 2), indicating that longer reaction times results in a higher degree of C—C bond cleavage.

TABLE 2
Product distribution in mass and mass balance: 2 g HDPE at 330° C.,
20 bar N₂ for different reaction times. Gasoline (C5-C12); Diesel (C9-C22)

| Catalyst | Toluene | C1-C4 | Gasoline | Diesel | C23+ | Solid | Mass balance |
|---|---|---|---|---|---|---|---|
| W/USY (4 hours) | 0.2 g | 8.86% | 67.11% | 52.06% | 3.89% | 7.10% | 89.1% |
| W/USY (6 hours) | 0.2 g | 11.90% | 63.34% | 53.40% | 4.53% | 5.66% | 92.6% |

TABLE 3
Product distribution in mass and mass balance: 2 g HDPE at 330°
C., 30 bar N₂ over HY (30) catalyst. Gasoline (C5-C12); Diesel (C9-C22)

| Catalyst | Toluene | C1-C4 | Gasoline | Diesel | C23+ | Solid | Mass balance |
|---|---|---|---|---|---|---|---|
| HY (30) | 0 | 12.10% | 54.69% | 35.42% | 0.33% | 22.22% | 92.5% |
| HY (30) | 0.2 g | 10.52% | 61.82% | 44.51% | 0.81% | 12.21% | 90.3% |

Table 3 and FIG. 3 highlight the effect of the hydrocarbon promoter when added to a zeolite catalyst without W or Re. The data indicates that the presence of the hydrocarbon promoter enhances C—C bond cleavage in HDPE giving higher overall decomposition and gasoline production. Comparatively, the incorporation of W into the USY zeolite (W/USY) gave slightly higher gasoline yield (67.11%) with 0.2 g toluene albeit with increased liquid product (46% in mass), which is thought to correspond to substituted aromatic compounds (see FIG. 4). The formation of substituted aromatic products, such as multi methylbenzene, suggests that toluene can scavenge CH₂ moieties from the polyolefin. It is thought that this occurs via toluene alkylation of the polyolefin over the BAS of the aluminosilicate at elevated temperatures. It is also thought that the removal of an electron from the aromatic anchored polyolefin over the BAS could favourably lead to the formation of a radical cation, a species capable of undergoing β-scission of C—C bonds to induce fragmentation of the polymer. It is widely reported that β-scission of C—C bonds is an essential step in the side-chain oxidation of alkyl aromatics and related compounds (Lai, et al. 2018., Ratkiewicz, 2011., Zhang et al. 2017).

TABLE 4
The final product distribution from 2 g HDPE over at 300° C., 20 bar
N₂ after 4 h over 0.2 g SiO₂—Al₂O₃ (with and without an acid wash) catalyst

| Catalyst | Toluene | | C1-C4 | Gasoline | Diesel | C23+ | Solid | Mass balance |
|---|---|---|---|---|---|---|---|---|
| SiAl (H+) | 0.2 | g | 10.55% | 60.91% | 55.27% | 0.82% | 6.25% | 94.9% |
| SiAl (H+) | 0 | g | 6.58% | 42.77% | 33.77% | 0.50% | 36.96% | 93.9% |
| SiAl | 0.2 | g | 6.94% | 43.33% | 32.37% | 0.29% | 37.60% | 93.4% |

Table 4 and FIG. 5 demonstrate the effects of a hydrocarbon promoter when added to a non-zeolite catalyst (SiO₂/Al₂O₃ with and without an acid wash) at a lower temperature of 300° C. Interestingly, the hydrocarbon promoter had a similar effect on C—C bond cleavage in HDPE, giving higher overall decomposition and gasoline production (60.91%) over the acid washed aluminosilicate catalyst. FIG. 6 indicates that the liquid product comprises less substituted aromatic compounds when a non-zeolite acidic catalyst is used (38% in mass). Interestingly, the hydrocarbon promoter did not have as much of an effect on the SiO₂—Al₂03 catalyst without an acid wash indicating the need for BAS. This is further supported by FIG. 7 which highlights the importance of both the hydrocarbon promoter and BAS. Nearly no pink peak is present at around 450° C. (indicative of the starting waste plastic) for pre-treated (i.e., acid washed) SiO₂/Al₂O₃ with toluene, implying that nearly all the waste plastic has been decomposed into smaller fractions. A large pink peak is observed at around 450° C. for the systems i) without toluene (10%/min) and ii) without reactivation of BAS (i.e., no acid wash) (6%/min). Similar conclusions can be drawn from analysing the GPC results (FIG. 8 and Table 5). Lower molecular weight and lower polydispersity index (Mw/Mn) are observed with hydrocarbon promoted SiO₂/Al₂O₃ (with acid wash). Conversely, without the promotion with toluene and in without BAS, higher molecular weights are observed along with broader solid product distribution (indicated by a higher polydispersity index), highlighting the importance of both hydrocarbon promotion and BAS in the efficient and selective decomposition of waste plastics.

TABLE 5
GPC results for the SiO₂—Al₂O₃ catalyst (solid residue).
2 g HDPE at 300° C., 20 bar N₂ after 4 h.

| | | | | |
|---|---|---|---|---|
| Catalyst | Toluene | Mw(g/mol) | Mn(g/mol) | Mw/Mn |

| | | | | | |
|---|---|---|---|---|---|
| SiAl (H+) | 0.2 | g | 300 | 200 | 1.44 |
| SiAl (H+) | 0 | g | 500 | 500 | 3.67 |
| SiAl | 0.2 | g | 400 | 400 | 4.98 |

Table 6 and FIG. 9 show the effect of increasing the temperature to 330° C. It is noted that the difference in gasoline yield when comparing reactions with and without hydrocarbon promotion is reduced due to the predominant thermal cracking effect. It is noted, however, that the higher diesel fraction and lower solid fraction proves that the hydrocarbon promoter enhances polyolefin decomposition. Reducing the reaction time under the same conditions increases the difference in gasoline yield indicating that lower temperatures and shorter reaction times promote the hydrocarbon promoted polyolefin decomposition reaction to form gasoline products (Table 7 and FIG. 10).

TABLE 6
The final product distribution from 2 g HDPE at 330° C., 20
bar N₂ after 4 h over 0.2 g SiO₂—Al₂O₃ (with acid wash).

| Catalyst | Toluene | | C1-C4 | Gasoline | Diesel | C23+ | Solid | Mass balance |
|---|---|---|---|---|---|---|---|---|
| SiAl (H+) | 0.2 | g | 12.04% | 61.17% | 41.61% | 0.10% | 13.87% | 92.0% |
| SiAl (H+) | 0 | g | 11.81% | 60.03% | 37.59% | 0.08% | 15.84% | 89.7% |

TABLE 7
The final product distribution from 2 g HDPE at 330° C., 20
bar N₂ after 2 h over 0.2 g SiO₂—Al₂O₃ (with acid wash).

| Catalyst | Toluene | | C1-C4 | Gasoline | Diesel | C23+ | Solid | Mass balance |
|---|---|---|---|---|---|---|---|---|
| SiAl (H+) | 0.2 | g | 11.28% | 66.70% | 41.14% | 0.12% | 9.57% | 92.7% |
| SiAl (H+) | 0 | g | 13.49% | 60.74% | 44.38% | 0.09% | 9.76% | 93.2% |

FIG. 11 highlights the effect of promotion with a different hydrocarbon promoter (decane). The effect of the hydrocarbon promoter was explored using a WO_{×}@SiO₂—Al₂O₃ (cross metathesis catalyst) and PtSn@SiO₂—Al₂O₃(hydrogenation-dehydrogenation catalyst) catalyst mixture. From this investigation it was observed that promotion with decane resulted in gasoline as the major product, showing very high selectivity for this fraction. This indicates that decane is a viable alternative to toluene as a hydrocarbon promoter for the selective decomposition of a polyolefin. Furthermore, the recyclability of the system was investigated by adding fresh HDPE in each cycle without adding any more aluminosilicate catalyst or hydrocarbon promoter. With each addition of fresh HDPE (indicated by cycles 2-4 in FIG. 11), the selective decomposition of the polyolefin to gasoline as well as the overall decomposition of the polyolefin remained high. This further confirms that the addition of a hydrocarbon promoter to an aluminosilicate catalyst facilitates efficient and selective polyolefin decomposition.

Under a H₂ atmosphere, it is clear from FIGS. 11 and 12 that the product distribution shifts toward the smaller hydrocarbon range with a greater degree of branching alkanes (no significant olefinic products are observed) along with the sharp reduction of n-decane. This demonstrates the effectiveness of polyolefin decomposition under a H₂ atmosphere in addition to the N₂ atmosphere also investigated. FIG. 13 also shows that increasing the H₂ pressure suppresses carbon deposition onto the aluminosilicate catalyst.

While specific embodiments of the invention have been described herein for the purpose of reference and illustration, various modifications will be apparent to a person skilled in the art without departing from the scope of the invention as defined by the appended claims.

### REFERENCES

Aisien, E. T., Otuya, I. C. & Aisien, F. A., 2021. Thermal and catalytic pyrolysis of waste polypropylene plastic using spent FCC catalyst. Environmental Technology & Innovation, Volume 22, p. 10455.

Chen, H., Wan, K., Zhang, Y. & Wang, Y., 2021. Waste to Wealth: Chemical Recycling and Chemical Upcycling of Waste Plastics for a Great Future. ChemSusChem, Volume 14, pp. 1-15.

Czajczynska, D. et al., 2018. Potentials of pyrolysis processes in the waste management sector. Energy Procedia, Volume 3, pp. 171-197.

Dutta, N. & Gupta, A., 2021. An experimental study on conversion of high-density polyethylene and polypropylene to liquid fuel. Clean Techn Environ Policy.

Haque, M. S., Uddin, S., Sayem, S. M. & Mohib, K. M., 2021. Coronavirus disease 2019 (COVID-19) induced waste scenario: A short overview. Journal of Environmental Chemical Engineering, 9(1), p. 104660.

Idumah, C. I., 2021. Recent advancements in thermolysis of plastic solid wastes to liquid. Journal of Thermal Analysis and Calorimetry.

Jiao, X. et al., 2021. Conversion of Waste Plastics into Value-Added Carbonaceous Fuels under Mild Conditions. Advanced Materials, Issue 2050192, pp. 1-9.

Kunwar, B. et al., 2017. Catalytic Thermal Cracking of Postconsumer Waste Plastics to Fuels. 2. Pilot-Scale Thermochemical Conversion. Energy & Fuels, 31(3), pp. 2705-2715.

Lai, L., Gudiyella, S., Liu, M., Green, W. H. 2018. Chemistry of Alkylaromatics Reconsidered. Energy Fuel. 32(4), 5489-5500. Liu, S. et al., 2021. Plastic waste to fuels by hydrocracking. Science Advances, 7(17), pp. 1-9.

Ratkiewicz, A. 2011. Kinetics of the C—C bond beta scission reactions in alkyl radicals. Phys. Chem. Chem. Phys. 13(33), 15037-15046. Vollmer, I. et al., 2021. Plastic Waste Conversion over a Refinery Waste Catalyst. Angewandte Chemie, 60(29), pp. 16101-16108.

Zhang, F. et al., 2021. Current technologies for plastic waste treatment: A review. Journal of Cleaner Production, Volume 282, p. 124523.

Zhang, Y., Cao, C., Li, Y., Yuan, W., Yang, X., Yang, J., Qi, F., Huang, T. P., Lee, Y. Y. 2017. Pyrolysis of n-butylbenzene at various pressures: influence of long side-chain structure on alkylbenzene pyrolysis. Energy Fuel. 31(12), 14270-14279.

1. A process for the catalytic decomposition of a polyolefin, the process comprising a step of contacting a polyolefin with:
(a) an aluminosilicate, and
(b) a hydrocarbon at a temperature of at least 200° C. and under an inert atmosphere,
wherein the hydrocarbon comprises 2-20 carbon atoms and the aluminosilicate comprises a plurality of Brønsted acid sites.

2. The process of claim 1, wherein the polyolefin comprises greater than 80 wt % of polyethylene, polypropylene or a combination thereof.

3. The process of claim 1, wherein the polyolefin is polyethylene, polypropylene or a combination thereof.

4. The process of claim 1, 2 or 3, wherein the polyolefin is selected from the group consisting of high density polyethylene (HDPE), low density polyethylene (LDPE), linear low-density polyethylene (LLDPE), high density polypropylene (HDPP), low density polypropylene (LDPP), linear low-density polypropylene (LLDPP) and a combination of two or more thereof.

5. The process of any one of the preceding claims, wherein the polyolefin is selected from the group consisting of high density polyethylene (HDPE), low density polyethylene (LDPE), high density polypropylene (HDPP) and a combination of two or more thereof.

6. The process of any one of the preceding claims, wherein the aluminosilicate is crystalline (e.g. a zeolite) or amorphous.

7. The process of any one of the preceding claims, wherein the aluminosilicate is selected from the group consisting of Y zeolite, ultrastable Y zeolite and amorphous SiO₂—Al₂O₃, each of which having a plurality of BAS.

8. The process of any one of the preceding claims, wherein the aluminosilicate comprises one or more transition metal promoter.

9. The process of claim 8, wherein the one or more transition metal promoters are selected from the group consisting of W, Re, Pt, Sn, Ir and Co.

10. The process of any one of the preceding claims, wherein the hydrocarbon is an aromatic compound comprising 5-20 carbon atoms.

11. The process of any one of the preceding claims, wherein the hydrocarbon has a molecular weight of less than 250 g mol⁻¹.

12. The process of any one of the preceding claims, wherein the hydrocarbon has a molecular weight of less than 200 g mol⁻¹.

13. The process of any one of the preceding claims, wherein the hydrocarbon has a molecular weight of less than 175 g mol⁻¹.

14. The process of any one of the preceding claims, wherein the hydrocarbon has a molecular weight of less than 150 g mol⁻¹.

15. The process of any one of the preceding claims, wherein the hydrocarbon is benzene, optionally substituted with one or more substituents independently selected from (1-5C)alkyl, (2-5C)alkenyl and (2-5C)alkynyl.

16. The process of any one of claims 1 to 9, wherein the hydrocarbon is selected from the group consisting of ethene, propene, butene, ethane, propane, butane, pentane, hexane, heptane, nonane, decane, benzene, toluene, xylene, cumene, mesitylene, 1,2,4,5-tetramethyl benzene and naphthalene

17. The process of claim 16, wherein the hydrocarbon is selected from the group consisting of benzene, toluene, xylene, cumene, mesitylene, 1,2,4,5-tetramethyl benzene and naphthalene.

18. The process of claim 16, wherein the hydrocarbon is toluene or decane.

19. The process of any one of the preceding claims, wherein the step of contacting the polyolefin with the aluminosilicate and the hydrocarbon is conducted at a temperature of 300-450° C.

20. The process of any one of the preceding claims, wherein the step of contacting the polyolefin with the aluminosilicate and the hydrocarbon is conducted at a temperature of 300-400° C.

21. The process of any one of the preceding claims, wherein the step of contacting the polyolefin with the aluminosilicate and the hydrocarbon is conducted at a temperature of 300-350° C.

22. The process of any one of the preceding claims, wherein the step of contacting the polyolefin with the aluminosilicate and the hydrocarbon is conducted under an atmosphere of nitrogen and hydrogen.

23. The process of any one of the preceding claims, wherein the step of contacting the polyolefin with the aluminosilicate and the hydrocarbon is conducted under an atmosphere of hydrogen.

24. The process of any one of the preceding claims, wherein the weight ratio of aluminosilicate to hydrocarbon is 1: (0.1-10).

25. The process of any one of the preceding claims, wherein the weight ratio of aluminosilicate to hydrocarbon is 1: (0.5-5).
